(19) United States
(12) Patent Application Publication
(10) Pub. No.: US 2009/0312384 A1
(43) Pub. Date: Dec. 17, 2009

(54) Benzimidazole As Cooling Compounds

(76) Inventors: Stefan Michael Furrer, Cincinnati, OH (US); Karen Ann Bell, Loveland, OH (US); Christophe C. Galopin, Chesterfield, VA (US); Thomas Scott McCluskey, Amelia, OH (US); Jay Patrick Slack, Loveland, OH (US)

Correspondence Address: CURATOLO SIDOTI CO., LPA, 24500 CENTER RIDGE ROAD, SUITE 280, CLEVELAND, OH, 44145, US

(21) Appl. No.: 12/307,835

(22) PCT Filed: Jul. 16, 2007
(86) PCT No.: PCT/CH07/00340

§ 371 Date: Jan. 7, 2009

Related U.S. Application Data
(60) Provisional application No. 60/830,964, filed on Jul. 14, 2006.

(51) Int. Cl. A61K 31/4184; C07D 235/12

(52) U.S. Cl. 514/394; 548/310.1

(57) ABSTRACT

A method of providing a cooling sensation to the skin or mucous membranes of the mouth by applying thereto a quantity of at least one chemical compound sufficient to cause a desirable degree of cooling sensation, the chemical compound comprising a compound of formula I:

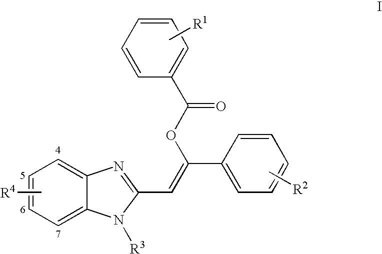

in which R¹, R² are independently in either the meta- or para-position and independently comprise at least one of hydrogen, halide, C₁-C₃ alkyl (linear or branched), halide, C₁-C₃ alkoxy, nitro, nitrile, amide or ester; R³ comprises at least one of C₁-C₅ alkyl or C₁-C₅ alkenyl groups (linear or branched), R⁴ comprises at least one of hydrogen, C₁-C₃ alkyl (linear or branched), or a halide. The cooling effect provided is in some instances superior to that achievable by any of the known commercial cooling agents.

Disclosed are cooling compounds, that is, compounds that give the sensation of cooling when applied to the skin or taken orally. Many such compounds are known to the art.

There has been discovered a new class of cooling compounds. There is disclosed herein a method of providing a cooling sensation to the skin or mucous membranes of the mouth by applying thereto a quantity of at least one chemical compound sufficient to cause a desirable degree of cooling sensation, the chemical compound comprising a compound of formula I:

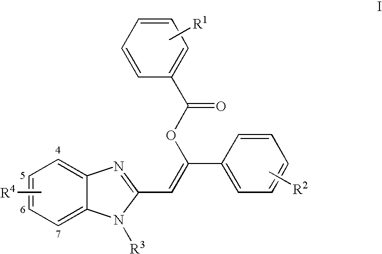

in which R¹, R² are independently in either the meta- or para-position and independently comprise at least one of hydrogen, halide, C₁-C₃ alkyl (linear or branched), halide, C₁-C₃ alkoxy, nitro, nitrile, amide or ester;

R³ comprises at least one of C₁-C₅ alkyl or C₁-C₅ alkenyl groups (linear or branched); and

R⁴ comprises at least one of hydrogen, C₁-C₃ alkyl (linear or branched) or halide.

In certain embodiments, R¹, R² independently comprise at least one of methoxy, chloro, bromo, fluoro, methyl, nitro or hydrogen.

In certain embodiments, R³ comprises at least one of C₁-C₃ alkyl or C₁-C₃ alkenyl groups, in particular, iso-propyl, propyl, methyl or allyl.

In certain embodiments, R⁴ comprises at least one moiety of methyl, bromine, or hydrogen in position 6 or 5.

Some of the compounds hereinabove described are novel. There is therefore also provided a compound of the formula I

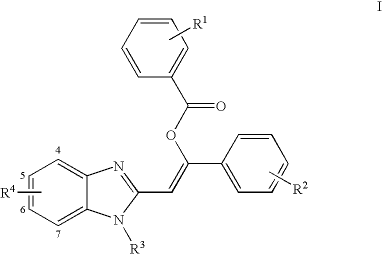

in which

R¹ and R² are independently selected from chloride and C₁-C₃ alkoxy groups,

R⁴ is C₁-C₃ alkyl (linear or branched), in certain embodiments methyl in position 5 or 6, and

R³ is C₁-C₃ alkyl, or C₁-C₃ alkenyl group, in certain embodiments iso-propyl, propyl, methyl or allyl.

In another embodiment with respect to the novel compounds, the alkoxy group from which R¹ and R² are selected is methoxy. In a further embodiment, R¹ and R² are either both methoxy, or one is methoxy and the other chloride.

The cooling compounds may be added to compositions applied to the skin or mucous membranes of the mouth to provide a cooling sensation. By “applying” is meant any form of bringing into contact, for example, oral ingestion in solid, liquid or spray form, or, in the case of tobacco products, inhalation. In the case of application to the skin, it may be, for example, by including the compound in a cream or salve, or in a sprayable composition. There is therefore provided a composition for oral, nasal or topical application, comprising a cooling amount of at least one chemical compound as hereinabove described.

Tn the term “composition” is included any kind of commercial product in which the compounds hereinabove described may be desired to be used. In particular, there is provided a product selected from the group consisting of topical products, oral care products, nasal care products, toilet articles, ingestible products and chewing gum, which product comprises a product base and an effective amount of at least one cooling compound of formula (I).

Products include foodstuffs and beverages of all kinds; confectionery products, for example, candies, mints and gums; edible films of all kinds; medicinal preparations in solid, liquid or spray form for oral, nasal and topical use; toothpastes and tooth gels; mouthwashes; skin lotions; cosmetic creams; tobacco products and aerosol products. Apart from the cooling compounds described herein, such products are otherwise entirely conventional in their formulation, and all of the known standard ingredients may be used in art-recognised quantities. Thus, for example, in the case of a medicinal product, the ingredients is that substance, or are those substances, that confer the particular medicinal nature of the product on it, and are selected from the substances known to provide the desired medicinal effect.

In addition, all the normal auxiliary ingredients needed to make a commercial product in a desired form meay be used. Non-limiting examples of such ingredients include pigments, dyestuffs and coloring matters; surfactants and emulsifiers; rheology-modifying agents; thickening agents; fillers and extenders; solvents and diluents; antioxidants; preservative materials, foam stabilizers; and the like.

The incorporation of the cooling compounds in such products is also entirely conventional and can be achieved by the standard methods of the art. Thus, for example, they can be incorporated by mixing directly into a product base, or into a premix, which is later incorporated into a product. They may also be incorporated by any kind of encapsulation method and utilising any of the known and commercially-successful technologies, for example, granulation, spray-drying, coating, coacervation, and the like. Any suitable encapsulation material may be used.

The subject compounds and method of use give a superior cooling effect, at least comparable with the best commercial cooling agents. It is of course possible and permissible to blend two or more subject compounds. In addition, conventional cooling agents known to the art may also be used in conjunction with the compositions described above. These include menthol, menthone, isopulegol, N-ethyl p-menthanecarboxamide (WS-3), N,2,3-trimethyl-2-isopropylbutanamide (WS-23), menthyl lactate, menthone glycerine acetal (Frescolat® MGA), mono-menthyl succinate (Physcool®), mono-menthyl glutarate, O-menthyl glycerine (CoolAct® 10) and 2-sec-butylcyclohexanone (Freskomenthe®).

The subject compounds and methods are now further described with reference to the following non-limiting examples.

### EXAMPLE 1

### General Preparation:

A) Preparation of 2-methyl-1-alkyl 1H-benzo[d]imidazole, Example 2-methyl-1-propyl-1H-benzo[d]imidazole:

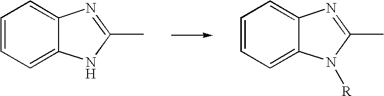

In a 500 mL flask, fitted with magnetic stirrer, 33 g of potassium hydroxide pellets (86%) [0.50 mol], 13.22 g methylbenzimidazole and 250 mL of acetone were added. 20 g of propyliodide was then added and the mixture was stirred at room temperature for 3 h. The mixture was extracted with 2× methyl tert.-butyl ether and water. The org. layers were washed with brine, dried over MgSO₄ and concentrated. The crude product purified by column chromatography yields 15.3 g of brown oil (88% yield).

¹H NMR (300 MHz; CDCl₃) δ: 7.7 (m, 1H) 7.2 (m, 3H), 4.04 (dd, 2H), 2.60 (d, 3H), 1.83 (m, 2H), 0.96 (dd, 3H)

¹³C NMR (300 MHz; CDCl₃) δ: 151.4, 142.7, 135.2, 121.8, 121.6, 119.0, 109.2, 45.3, 23.0, 13.9, 11.4

2-methyl-1-isopropyl-1H-benzo[d]imidazole:

¹H NMR (300 MHz; CDCl₃) δ: 7.7-7.5 (m, 1H) 7.2 (m, 3H), 4.65 (m, 1H), 2.61 (s, 3H), 1.62 (m, 6H)

¹³C NMR (300 MHz; CDCl₃) δ: 150.9, 143.1, 133.8, 121.8, 121.5, 119.2, 111.1, 48.0, 21.4, 15.0, 14.9

B) Preparation of Ketols from 1-alkyl-2-methyl-1H-benzo[d]imidazoles:

2-methyl-1-isopropyl-1H-benzo[d]imidazoIe was added to a round bottom flask with a N₂ inlet, magnetic stirring bar and a thermocouple and dissolved in THF (4 mL/mmol). The resulting mixture was cooled to −78° C. using dry ice/isopropanol bath and Lithium diisopropylamide (2M solution in THF/n-heptane) (1.1 eq.) was added slowly using a syringe while maintaining the temperature <−60° C. The reddish reaction mixture was allowed to stir at −78° C. for about 2 h and a solution of methyl benzoate (1.5 eq) in THF (1 mL/mmol) was added slowly over a period of 10 min, using a syringe. The yellowish colored solution was slowly allowed to warm to room temperature and the progress of the reaction was monitored by TLC. After the reaction was complete, the mixture was cooled using ice/water bath and saturated aqueous NH₄Cl (4 mL/mmol) and MTBE (4 mL/mmol) was added. The organic layer was separated, washed with water and concentrated under vacuum at −35° C. The residue was purified on silica gel using ethyl acetate/heptane.

### C) Preparation of Benzimiazole-Based Analogs:

Ketol was added to a round bottom flask with a N₂ inlet, magnetic stirring bar and a thermocouple and dissolved in toluene (5 mL/mmol). To the mixture triethylamine (1.5 eq) was added. The resulting mixture was cooled to 0° C. using ice/water bath and benzoyl chloride (1.2 eq.) was added slowly using a syringe while maintaining internal temperature <10° C. The dark colored solution was slowly allowed to warm to room temperature and the progress of the reaction was monitored by TLC. After the reaction was complete, the mixture was cooled using ice/water bath and saturated aqueous NaHCO₃ was added. The organic layer was separated, washed with water and concentrated under vacuum at ˜35° C. The residue was stirred with ethyl acetate/heptane to give a solid suspension. The solid was collected by filtration and washed with heptane and dried.

### EXAMPLES 2-12

Using the method described in Example 1, a number of compounds were prepared.

EXAMPLE 2

1-(4-chlorophenyl)-2-(1-isopropyl-5-methyl-1H-benzo[d]imidazol-2-yl)vinyl 4-methoxybenzoate

¹H NMR (300 MHz; CDCl₃) δ: 8.2 (d, 2H) 7.6 (d 2H), 7.4-7.3 (m, 4H), 7.0 (m, 4H), 4.9 (m, 1H), 3.9 (s, 3H). 2.4 (s, 3H), 1.6 (d, 6H)

¹³C NMR (300 MHz; CDCl₃) δ: 164.0, 163.8, 145.9, 135.7, 133, 132.7, 131.9, 130.5, 128.9, 126.7, 124.2, 121.8, 119.4, 113.7, 111.1, 103.6, 55.5, 48.4, 21.7, 21.3

LC−MS (ESI+), m/z 461 [M+]

MP: 155-160° C.

### EXAMPLE 3

2-(1-isopropyl-5-methyl-1H-benzo[d]imidazol-2-yl)-1-(4-methoxyphenyl)vinyl 4-methoxybenzoate

¹H NMR (300 MHz; CDCl₃) δ: 8.3-8.1 (m, 2H) 7.7-7.5 (m 2H), 7.4-7.2 (m, 2H), 7.1-6.8 (m, 6H), 5.0-4.8 (m, 1H), 4.0-3.6 (m, 6H). 2.6-2.2 (m, 3H), 1.8-1.4 (m, 6H)

¹³C NMR (300 MHz; CDCl₃) δ: 164.1, 163.6, 160.8, 152.3, 145.7, 132.5, 131.3, 130.8, 127.2, 126.9, 123.9, 121.7, 122.0, 119.5, 114.1, 113.6, 110.8, 108.4, 101.4, 55.4, 55.3, 48.1, 21.6, 21.2

LC−MS (ESI+), m/z 457 [M+]

MP: 147-152° C.

### EXAMPLE 4

1-(4-chlorophenyl)-2-(1-isopropyl-6-methyl-1H-benzo[d]imidazol-2-yl)vinyl 4-chlorobenzoate

¹H NMR (300 MHz; CDCl₃) δ: 8.3 (d, 2H) 7.6 (d 2H), 7.5-7.4 (m, 4H), 7.2-7.0 (m, 4H), 5.1-5.0 (m, 1H), 2.4 (s, 3H), 1.7 (d, 6H)

¹³C NMR (300 MHz; CDCl₃) δ: 163.3, 161.2, 144.2, 141.3. 140.3, 136.1, 134.2, 132.1, 131.8, 129.3, 128.9, 128.7, 127.1, 126.7, 125.4, 117.4, 112.0, 101.3, 49.6, 21.9, 21.5

LC−MS (ESI+), m/z 465 [M+]

MP: 158-165° C.

### EXAMPLE 5

2-(1-isopropyl-6-methyl-1H-benzo[d]imidazol-2-yl)-1-(4-methoxyphenyl)vinyl 4-methoxybenzoate

¹H NMR (300 MHz; CDCl₃) δ: 8.2 (d, 2H) 7.6 (d 2H), 7.4 (d, 1H), 7.0-6.8 (m, 7H), 5.0-4.8 (m, 1H), 3.9 (s, 3H), 3.8 (s, 3H), 2.4 (s, 3H), 1.7 (d, 6H)

¹³C NMR (300 MHz; CDCl₃) δ: 164.2, 163.6, 160.8, 152.5, 146.3, 132.9, 132.6, 132.1, 127.1, 126.9, 123.4, 122.1, 119.2, 114.1, 113.5, 111.2, 101.3, 55.4, 55.2, 48.1, 21.9, 21.6

LC−MS (ESI+), m/z 457 [M+]

MP: 125-130° C.

### EXAMPLE 6

1-(4-chlorophenyl)-2-(1-propyl-1H-benzo[d]imidazol-2-yl)vinyI 4-chlorobenzoate

¹H NMR (300 MHz; DMSO) δ: 8.2 (d, 2H) 7.9 (d 2H), 7.7 (d, 2H), 7.6-7.5 (m, 3H), 7.4 (s, 1H), 7.2-7.1 (m, 2H), 4.4 (t, 2H), 1.9-1.7 (m, 2H), 0.9 (t, 3H)

### EXAMPLE 7

1-(4-fluorophenyl)-2-(1-methyl-1H-benzo[d]imidazol-2-yl)vinyl 4-fluorobenzoate

¹H NMR (300 MHz; DMSO) 5:8.3-8.2 (m, 2H) 7.9 (m 2H), 7.5 (d, 1H), 7.4 (t, 2H), 7.35 (t, 1H), 7.3 (t, 2H), 7.2 (t, 1H), 7.1-7.0 (m, 2H), 3.95 (s, 3H)

### EXAMPLE 8

2-(1-methyl-1H-benzo[d]imidazol-2-yl)-1-p-tolylvinyl 4-methylbenzoate

¹H NMR (300 MHz; DMSO) δ: 8.05 (d, 2H) 7.75 (d, 2H), 7.5 (d, 1H), 7.45 (d, 2H), 7.3 (m, 3H), 7.25-7.05 (m, 3H), 3.95 (s, 3H), 2.5 (s, 3H), 2.4 (s, 3H)

### EXAMPLE 9

1-(4-chlorophenyl)-2-(1-isopropyl-1H-benzo[d]imidazol-2-yl)vinyl 4-chlorobenzoate

¹H NMR (300 MHz; DMSO) δ: 8.15 (d, 2H) 7.9 (d, 2H), 7.7 (t, 3H), 7.55 (d, 2H), 7.5 (s, 1H), 7.2-7.05 (m, 3H), 5.2 (m, 1H), 1.6 (d, 6H)

### EXAMPLE 10

1-(4-chlorophenyl)-2-(1-isopropyl-5-methyl-1H-benzo[d]imidazol-2-yl)vinyl 4-chlorobenzoate

¹H NMR (300 MHz; CDCl₃) δ: 8.2 (d, 2H), 7.6 (d 2H), 7.5 (d, 2H), 7.4 (m, 3H), 7.2 (s, 1H), 7.0 (d, 1H), 6.9 (s, 1H), 4.9-4.8 (m, 1H), 2.4 (s, 3H), 1.7 (d, 6H)

¹³C NMR (300 MHz; CDCl₃) δ: 163.8, 150.6, 145.9, 139.6, 135.6, 133.1, 131.8, 131.5, 130.9, 129.2, 128.9, 128.6, 128.3, 126.5, 124.0, 119.8, 110.8, 103.6, 47.9, 21.7, 21.2

MP: 145-152° C.

### EXAMPLE 11

2-(1-isopropyl-5-methyl-1H-benzo[d]imidazol-2-yl)-1-(4-methoxyphenyl)vinyl 4-chlorobenzoate

¹H NMR (300 MHz; CDCl₃) δ: 8.3 (d, 2H), 7.6 (d 2H), 7.5 (m, 2H), 7.3 (m, 2H), 7.1-7.0 (m, 2H), 6.8 (d, 2H), 5.0-4.9 (m, 1H), 3.7 (s, 3H), 2.4 (s, 3H), 1.7 (d, 6H)

¹³C NMR (300 MHz; CDCl₃) δ: 163.3, 161.1, 145.7, 139.9, 132.0, 131.7, 130.0, 129.2, 128.7, 127.8, 127.0, 126.1, 124.5, 116.5, 114.8, 111.2, 109.0, 55.2, 48.7, 21.5, 21.2

MP: 160-166° C.

### EXAMPLE 12

1-(4-chlorophenyl)-2-(1-isopropyl-6-methyl-1H-benzo[d]imidazol-2-yl)vinyl 4-methoxybenzoate

¹H NMR (300 MHz; CDCl₃) δ: 8.3 (d, 2H), 7.6(m, 3H), 7.5 (d, 1H), 7.2 (s, 1H), 7.1 (m, 3H), 7.0 (d, 2H), 5.2-5.0 (m, 1H), 3.8 (s, 3H), 2.4 (s, 3H), 1.7 (d, 6H)

¹³C NMR (300 MHz; CDCl₃) δ: 164.1, 163.6, 154.2, 144.2, 136.2, 134.5, 133.1, 131.6, 128.7, 126.9, 125.8, 120.5, 117.0, 113.9, 112.2, 101.5, 55.4, 50.1, 21.9, 21.5

### EXAMPLE 13

Experiments on cooling properties/intensities.

A group of panelists was asked to taste various aqueous solutions of compounds of formula (I) and to indicate which solutions had cooling intensities similar or slightly higher than that of a solution of menthol at 2 ppm. The results are shown in Table 1.

TABLE 1
Experiments on cooling intensity

| Chemical | Concentration | Odor |
|---|---|---|
| Comparison: 1-Menthol | 2 ppm solution | Minty |
| N-ethyl p-menthanecarboxamide (WS-3) | 1.5 ppm | None |
| 1-(4-methoxyphenyl)-2-(1-methyl-1H- | 0.5 ppm | None |
| benzo[d]imidazol-2-yl)vinyl 4-methoxybenzoate | | |
| 2-(1-isopropyl-6-methyl-1H-benzo[d]imidazol-2-yl)- | 0.5 ppm | None |
| 1-(4-methoxyphenyl)vinyl 4-methoxybenzoate | | |
| (Z)-2-(1-isopropyl-5-methyl-1H-benzo[d]imidazol-2- | 0.5 ppm | None |
| yl)-1-(4-methoxyphenyl)vinyl 4-methoxybenzoate | | |
| (E)-2-(1-methyl-1H-benzo[d]imidazol-2-yl)-1-p- | 2.0 ppm | None |
| tolylvinyl 4-methylbenzoate | | |

### EXAMPLE 14

Application in toothpaste

| | |
|---|---|
| opaque toothgel | 99.50 g |
| cooling compound* as a 5% (w/w)solution in ethanol | 0.20 g |
| peppermint oil, terpeneless | 0.25 g |
| saccharin | 0.20 g |

*Compound of Example 5

The chemicals were mixed in the toothgel, a piece of toothgel was put on a toothbrush and a panelist's teeth were brushed. The mouth was rinsed with water and the water was spit out. An intense cooling sensation was felt by the panelist in all areas of the mouth. The cooling perception lasted for 90 minutes.

Although the invention has been described in detail through the above detailed description and the preceding examples, these examples are for the purpose of illustration only and it is understood that variations and modifications can be made by one skilled in the art without departing from the spirit and the scope of the invention. It should be understood that the embodiments described above are not only in the alternative, but can be combined.

1. A method of providing a cooling sensation to the skin or mucous membranes of the mouth by applying thereto a quantity of at least one chemical compound sufficient to cause a desirable degree of cooling sensation, the chemical compound comprising a compound of formula I: 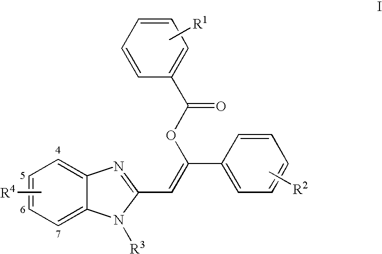
in which R¹, R² are independently in either the meta- or para-position and independently comprise at least one of hydrogen, halide, C₁-C₃ alkyl (linear or branched), halide, C₁-C₃ alkoxy, nitro, nitrile, amide or ester;
R³ comprises at least one of C₁-C₅ alkyl or C₁-C₅ alkenyl groups (linear or branched); and
R⁴ comprises at least one of hydrogen, C₁-C₃ alkyl (linear or branched), or a halide.

2. The method according to claim 1, in which R¹, R² independently comprise at least one moiety of methoxy, chloro, bromo, fluoro, methyl, nitro or hydrogen.

3. The method according to claim 1, in which R³ comprises at least one of C₁-C₃ alkyl or C₁-C₅ alkenyl groups.

4. The method according to claim 3, in which R³ comprises at least one of iso-propyl, propyl, methyl or allyl.

5. The method according to claim 1, in which R⁴ is located in either position 5 or 6 and comprises at least one of methyl, bromine or hydrogen.

6. A compound of the formula I 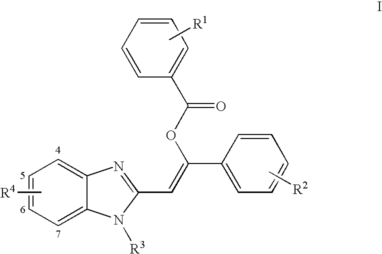
in which
R¹ and R² are selected from chloride and C₁-C₃ alkoxy groups,
R⁴ is C₁-C₃ alkyl (linear or branched), and
R3 is C₁-C₃ alkyl or C₁-C₅ alkenyl group.

7. A composition for oral, nasal or topical application comprising a cooling amount of at least one chemical compound comprising a compound of formula I: 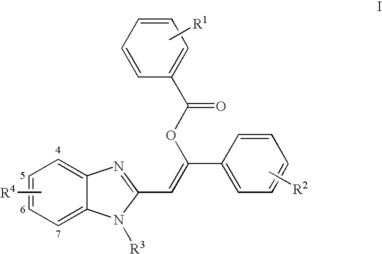
in which R¹, R² are independently in either the meta- or para-position and independently comprise at least one of hydrogen, halide, C₁-C₃ alkyl (linear or branched), halide, C₁-C₃ alkoxy, nitro, nitrile, amide or ester;
R³ comprises at least one of C₁-C₅ alkyl or C₁-C₅ alkenyl groups (linear or branched); and
R⁴ comprises at least one of hydrogen, C₁-C₃ alkyl (linear or branched) or halide.

8. The composition according to claim 7, in which R¹, R² independently comprise at least one moiety of methoxy, chloro, bromo, fluoro, methyl, nitro or hydrogen.

9. The composition according to claim 7, in which R³ comprises at least one of C₁-C₃ alkyl or C₁-C₅ alkenyl groups.

10. The composition according to claim 9, in which R³ comprises at least one of iso-propyl, propyl, methyl or allyl.

11. The composition according to claim 7, in which R⁴ is located in either position 5 or and comprises at least one of methyl, bromine or hydrogen.
